(19) United States
(12) Patent Application Publication
(10) Pub. No.: US 2026/0224322 A1
(43) Pub. Date: Aug. 6, 2026

(54) ADJUSTABLE HEIGHT RAIL-CLAMP SYSTEM FOR MOUNTING ROWS OF MODULAR HORIZONTAL TILT BINS

(71) Applicant: CareFusion 303, Inc., San Diego, CA (US)

(72) Inventors: Philip Pupa, San Diego, CA (US); Michael Becker, San Diego, CA (US); Palak Lee, San Diego, CA (US)

(21) Appl. No.: 19/158,379

(22) PCT Filed: Feb. 21, 2023
(86) PCT No.: PCT/US2023/062921

§ 371 Date: Aug. 20, 2025

(51) Int. Cl. A61B 50/10; A61B 50/30

(52) U.S. Cl. CPC A61B 50/10; A61B 2050/105; A61B 2050/3008; A61B 2050/3009

(57) ABSTRACT

A medical station comprises a base and an upper module formed of a structure positioned atop the base. At least one vertical mounting post is attached to at least one of the base and the upper module and a first mounting clamp is slidably mounted to the vertical mounting post. A horizontal rail is attached to the at least one vertical mounting post via the first mounting clamp, wherein a vertical position of the horizontal rail is adjustable via sliding the first mounting clamp along the horizontal rail and wherein a storage bin can be removably attached to the horizontal rail.

### TECHNICAL FIELD

The current subject matter described herein relates generally to a medical station such as an anesthesia cart. In particular, the current subject matter relates to a medical station having a storage bin system configured to be adjustably positioned relative to the medical station.

### BACKGROUND

Medical stations such as anesthesia carts may be incorporated in various clinical and healthcare environments. For example, an anesthesia cart includes a standing unit or base upon which one or more storage bins are removably mounted, such as by using hooks to attach the bins on horizontal rails attached to the anesthesia cart. The horizontal rails are in a fixed position on the anesthesia cart. This fixed configuration can be limiting on the size and types of bins that can be mounted to the anesthesia cart, which can be inconvenient for a user.

### SUMMARY

Aspects of the current subject matter relate to a medical station, such as an anesthesia cart, having a storage bin mounting system on which storage bins can be adjusted in position and size relative to the medical station. In an example, a position of one or more bins can be adjusted at least along a vertical direction. This can render a storage capability of the cart adjustable such as permitting variability in the size of bins as well as the quantity of bins. This renders the storage system more accessible than storage systems of a fixed storage size configuration.

In one aspect, there is disclosed a medical station, comprising: a base and an upper module formed of a structure positioned atop the base; at least one vertical mounting post attached to at least one of the base and the upper module; a first mounting clamp that is slidably mounted to the vertical mounting post; and a horizontal rail attached to the at least one vertical mounting post via the first mounting clamp, wherein a vertical position of the horizontal rail is adjustable via sliding the first mounting clamp along the horizontal rail, wherein a storage bin can be removably attached to the horizontal rail.

In another aspect, there is disclosed a storage system for a medical station, comprising: a first mounting post; a first mounting clamp slidably attachable to the first mounting post such that a position of the first mounting clamp along the first mounting post is adjustable; and a first mounting rail attachable to the first mounting post via the first mounting clamp, wherein a position of the first mounting rail relative to the first mounting post is adjustable via sliding the first mounting clamp along the first mounting post, wherein a storage bin can be removably attached to the first mounting rail.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a rear, perspective view of a medical station such as an anesthesia cart consistent with implementations of the current subject matter;

FIG. 2 is front, perspective view of an upper portion of the medical station consistent with implementations of the current subject matter;

FIG. 3 is rear, perspective view of an adjustable storage bin system of the medical station consistent with implementations of the current subject matter; and

FIG. 4 is an enlarged view of a clamp assembly of the storage bin system consistent with implementations of the current subject matter.

When practical, similar reference numbers denote similar structures, features, or elements.

### DETAILED DESCRIPTION

Aspects of the current subject matter relate to a medical station with position-adjustable storage bins system that permits flexibility in the size, shape and quantity of storage bins coupled to the medical station. The medical station may be a standalone module or a component of a modular system. The medical station may be part of a medication management system that includes various modules and stations for the secure storage and management of medication.

Aspects of the current subject matter provide a medical station formed of a structure, such as a pedestal structure, that forms a work station where a person can perform, monitor, or otherwise manage medical related work. In some implementations, the medical station is an anesthesia cart. In an implementation, the medical station has a base and an upper portion or module, where the upper module connects to or is otherwise integral with the base. The upper module is a structure or frame positioned atop the base. In some implementations, the upper module may connect to more than one type of base. In some implementations, the base is a standing unit or module. The base and the upper module may be used for a variety of purposes including to store medication, supplies, and/or documentation such as medical records or files in or on one or more storage bins. In some implementations, the base may include a refrigeration and/or freezer unit. The medical station may include electronic components, such as one or more processing devices, and other valuable or sensitive components.

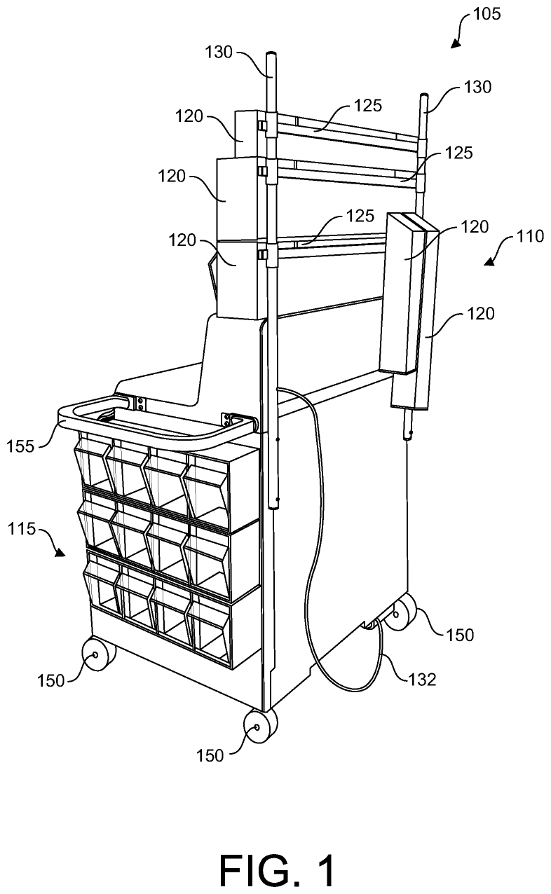

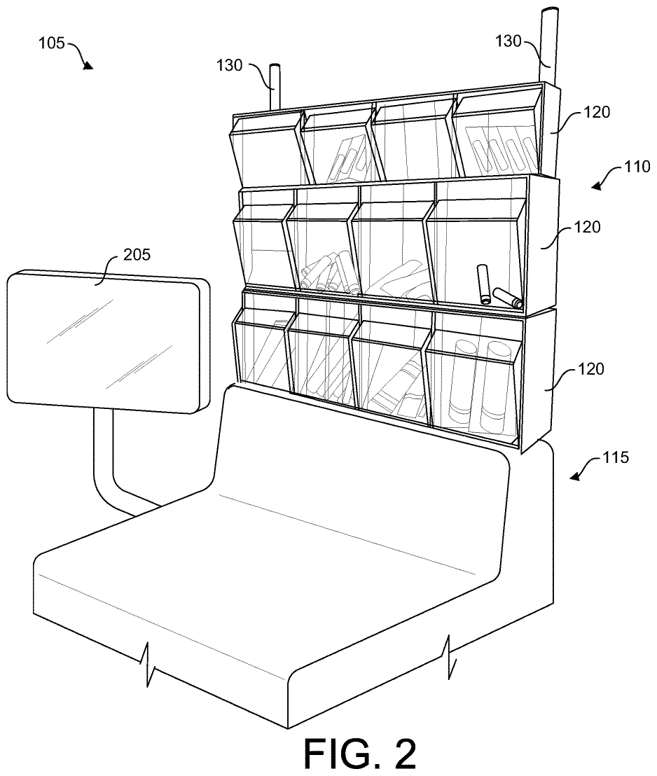

FIGS. 1-2 depict various aspects of a medical station 105 consistent with implementations of the current subject matter. As mentioned, in an example implementation, the medical station is an anesthesia cart although the type of medical station can vary. FIG. 1 depicts a rear, perspective view of the medical station 105, while FIG. 2 depicts a front, perspective view of an upper module or upper portion 110 of the medical station 105.

The upper portion 110 is shaped, configured and positioned on a base portion 115 (FIG. 1) that can be positioned on a surface such as a floor surface. The upper portion 110 includes one or more storage bins 120 that are removably mounted on respective mounting elements, such as horizontal mounting elements. In an implantation, the storage bins include or can be coupled to hook structures or other mechanical structures that enable the storage bins to be hung or otherwise coupled on one or more of the mounting elements. In an implementation, the mounting elements are elongated mounting rails 125, which are each positioned along a respective horizontal axes or an axes that has at least a horizontal component. The storage bins 120 can be mounted in a front facing orientation (as shown in FIG. 2) and/or in rear facing orientation (as shown in FIGS. 1 and 2.)

The mounting rails 125 are adjustably and movably attached to one or more vertical mounting posts 130 such that at least a vertical position of each mounting rail 125 along the vertical mounting posts 130 is adjustable, as described further below. For example, the mounting rails 125 can be configured to slidably move along a mounting post 130 and can also be locked in position relative to a mounting post. The adjustable aspect of the system permits a user to removably mount storage bins of various sizes on the medical station 105, as described more fully below. The vertical mounting posts 130 can be fixedly or removably attached to the medical station.

At least one mounting post 130 may be coupled to a power cord 132. The power cord 132 can be at least partially positioned inside one of the mounting posts 130 and electrically coupled to one or more components of at least one of the mounting rail 125 and storage bin 120. The power cord 132 may transfer or otherwise provide power from a power source (such as a wall outlet or a power source coupled to the medication station 105) to one or more storage bins 120 via at least one mounting rail 125. In some implementations, at least one specific mounting rails 125 is power enabled via the power cord 132 while another mounting rail 125 is not powered. In other implementations, all the mounting rails are powered. The power may be used by one or more of the storage bins 120 for lighting, device charging, locking, inventor control processing (e.g., locking or sensors), or other medication dispensing or administration activity associated with the storage bin 120.

The storage bins 120 can vary in configuration including for example size, shape and spatial arrangement. In an implementation, at least one storage bin 120 forms a pocket or container sized and shaped to receive and contain one or more items. The storage bins 120 can have an opening and/or a door to the opening, wherein a user can place on object into the storage bin or remove an object from the storage bin via the opening. The storage bins 120 can vary in size and shape. A collection of storage bins 120 can include multiple storage bins of similar or same size and shape as well as one or more storage bins of different size and shape relative to other storage bins in the collection of storage bins.

With reference still to FIGS. 1 and 2, the base portion 115 and/or the upper portion 110 can optionally include various components. For example, the base portion 115 can define a flat or contoured work surface for a healthcare worker. The work surface can have a recessed configuration to hold or contain items (such as medications, pens, papers, or other items.) A display unit 205 (FIG. 2) may couple to the medical station 105 (such as to the upper portion 110) to allow for the healthcare workers to access and use a processing device or any other aspect of the medical station. The base portion 115 may include one or more wheels 150 (FIG. 1) to enable a user to move the medical station along the floor such as by accessing a handle 155. The medical station 105 can further include any of a variety of other components, such as one or more power supplies and corresponding power cords.

As mentioned, a size and a shape of the storage bins 120 as well as a positioning of the storage bins 120 relative to the medical station can vary. With reference to the rear view of FIG. 1, a pair of storage bin 120 are mounted on a rear portion of the medical station 105 such that a user can access the storage bins 120 from a rear side of the medical station 105. The rear storage bins 120 are substantially rectangular, prismatic and elongated in shape although it should be appreciated that the shape of the storage bins 120 can vary.

FIG. 2 shows storage bins 120 mounted on a front portion of the medical station 105 wherein the storage bins are arranged in a series of three horizontal rows of the same or different vertical size. The quantity of storage bins in each row can vary, as can the number of rows. Each row can include one or more storage bins, wherein the storage bins in a row can be fixedly coupled to each other or removable from one another.

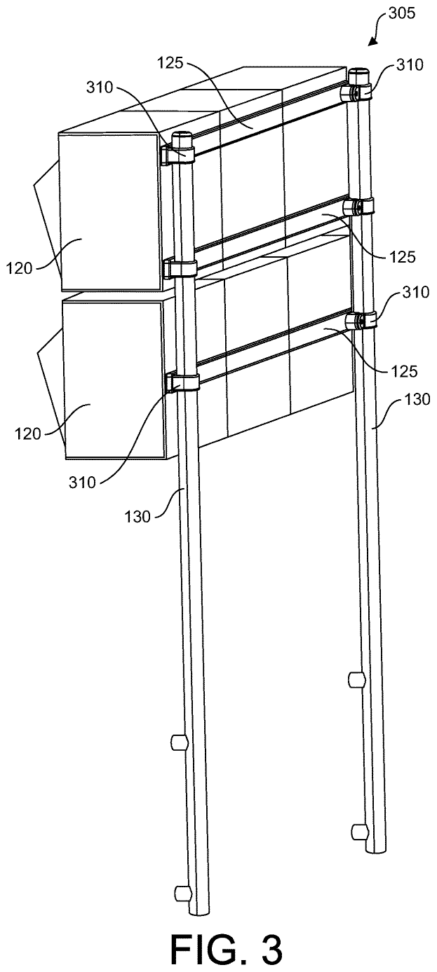

FIG. 3 shows an example implementation of a storage bin system 305 of the medical station 105. The storage bin system 305 is configured to permit the storage bins to be adjusted in position such as along a vertical direction. The storage bin system 305 includes at least one vertical mounting post 130, which can be removably or fixedly attached to the medical station. In the illustrated implementation, the storage bin system 305 includes two vertical mounting posts 130 that are arranged in a parallel or substantially parallel orientation relative to one another. The quantity of vertical mounting posts can vary. At least one horizontal mounting rail 125 is movably and removably mounted on the two vertical mounting posts 130 such as at opposite ends of the mounting rail 125. The mounting rail 125 provides a mechanism for removably attaching a respective storage bin 120 or a series of storage bins 120 such as by hooking and hanging the storage bin(s) 120 on a mounting rail 125. FIG. 3 shows the storage bins 120 mounted in a front orientation although, as mentioned, one or more storage bins can also be mounted in a rear orientation such as shown in FIG. 1.

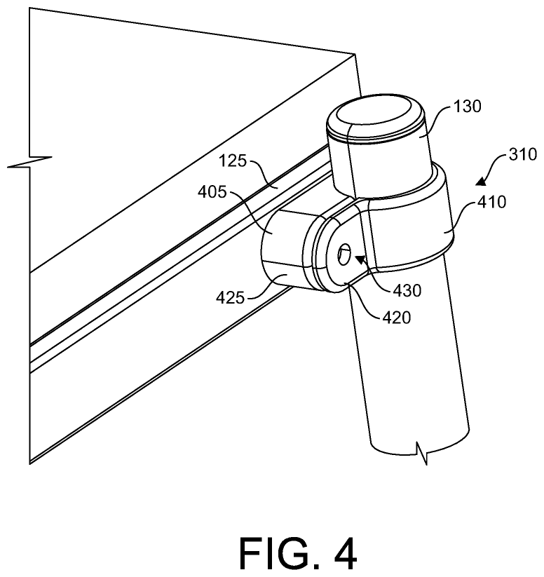

With reference to FIGS. 3 and 4, each mounting rail 125 includes a mounting clamp 310 that mechanically couples the mounting rail 125 to the vertical mounting post 130. The mounting clamps 310 couple to the vertical mounting post 130 such that the mounting clamp 310 (and attached mounting rail 125) can slide along the length of the vertical mounting post to thereby adjust a vertical position.

FIG. 4 shows an enlarged view of an example mounting clamp 310, which is a structure that wraps around a respective vertical mounting post 130. The mounting clamp 310 has a contact surface 405 that is fixedly or removably attached to the mounting rail 125. The mounting clamp 310 includes a mating structure 410 that defines an internal lumen, opening, or cavity sized and shaped to receive therein the vertical mounting post 130. The lumen, opening, or cavity can be selectively tightened or loosened to lock the mounting clamp 310 in place or to slide the mounting clamp 310 along the vertical mounting post 130.

In an embodiment, the lumen, opening, or cavity has a shape that substantially conforms to or complements an outer shape or contour of the vertical mounting post 130. This permits the mounting clamp 310 to slide along the vertical mounting post 130. The outer shape of the vertical mounting post 130 can vary. In an implementation, the vertical mounting post 130 is cylindrical although other shapes are within the scope of this disclosure.

The mounting clamp 310 can be loosened, opened or otherwise unlocked relative to the vertical mounting post 130 by separating a first portion 420 and a second portion 425 from one another to thereby loosen or release the mounting clamp relative to the vertical mounting post 130. Also, the mounting clamp 310 can be tightened and locked in position around the vertical mounting post 130 such as by inserting a screw or other tightening or locking element into an opening 430 to secure and tighten the first portion 420 relative to the second portion 425. In this manner, the mounting clamp 310 tightens around the vertical mounting post with sufficient force and friction to lock a position of the mounting clamp 310 on the vertical mounting post.

An inner portion of the mounting clamp 310 can be made of or coupled to a substance or material that enhances or encourages a secure fit between the vertical mounting post and the mounting clamp 310. Such a material or substance can be deformable. In addition, the inner portion of the mounting clamp 310 can also include a lubrication or be made of a lubricous material to facilitate sliding of the mounting clamp 310 along the vertical mounting post when the mounting clamp is loosened.

In use, a user can loosen the mounting clamps 310 of a respective mounting rail 125 and slide the mounting rail 125 along the vertical mounting posts 310 to thereby adjust a vertical position of the mounting rail 125 relative to the vertical mounting posts. Once the mounting rails 125 is at a desired vertical position relative to the vertical mounting post 130 and the medical station, the user can lock the mounting clamp 310 to the vertical mounting post to thereby lock the vertical position of the attached mounting rail 125. The user can couple any quantity of mounting rails to the vertical mounting posts at any of a variety of vertical locations along the vertical mounting posts 310. This provides the user with flexibility in mounting storage bins of various sizes and in various spatial arrangements on the medical station.

Although the disclosure, including the figures, described herein may describe and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A medical station, comprising:
a base and an upper module formed of a structure positioned atop the base;
at least one vertical mounting post attached to at least one of the base and the upper module;
a first mounting clamp that is slidably mounted to the vertical mounting post; and
a horizontal rail attached to the at least one vertical mounting post via the first mounting clamp, wherein a vertical position of the horizontal rail is adjustable via sliding the first mounting clamp along the horizontal rail, wherein a storage bin can be removably attached to the horizontal rail.

2. The medical station of claim 1, further comprising the storage bin.

3. The medical station of claim 1, wherein the first mounting clamp can be tightened relative to the vertical mounting post to lock a position of the first mounting clamp and the horizontal rail relative to the vertical mounting post.

4. The medical station of claim 1, wherein the first mounting clamp has an internal lumen that slidably receives the vertical mounting post.

5. The medical station of claim 4, wherein the internal lumen has a shape that complements an outer shape of the vertical mounting post.

6. The medical station of claim 1, wherein the vertical mounting post is cylindrical.

7. The medical station of claim 1, wherein the storage bin can be mounted in either a front facing orientation or a rear facing orientation on the horizontal rail.

8. The medical station of claim 1, wherein the at least one vertical mounting post includes a first vertical mounting post and a second vertical mounting post and further comprising a second mounting clamp, wherein the first mounting clamp is slidably mounted on the first vertical mounting post and the second mounting clamp is slidably mounted on the second vertical mounting post.

9. The medical station of claim 1, wherein the at least one vertical mounting post is removably attached to at least one of the base and the upper module.

10. The medical station of claim 1, wherein the first mounting clamp includes a tightening element configured to tighten the first mounting clamp around the at least one vertical mounting post.

11. The medical station of claim 10, wherein the tightening element is a screw.

12. The medical station of claim 1, wherein the medical station is an anesthesia cart.

13. The medical station of claim 1, further comprising a power cord coupled to the at least one vertical mounting post.

14. A storage system for a medical station, comprising:
a first mounting post;
a first mounting clamp slidably attachable to the first mounting post such that a position of the first mounting clamp along the first mounting post is adjustable;
a first mounting rail attachable to the first mounting post via the first mounting clamp, wherein a position of the first mounting rail relative to the first mounting post is adjustable via sliding the first mounting clamp along the first mounting post, wherein a storage bin can be removably attached to the first mounting rail.

15. The storage system of claim 14, further comprising:
a second mounting post;
a second mounting clamp attached to the first mounting rail and slidably attachable to the second mounting post such that a position of the first mounting rail is adjustable relative to both the first mounting post and the second mounting post.

16. The storage system of claim 14, wherein the first mounting clamp can be tightened relative to the first mounting post to lock a position of the first mounting clamp and the horizontal rail relative to the first mounting post.

17. The storage system of claim 14, wherein the first mounting clamp has an internal lumen that slidably receives the first mounting post.

18. The storage system of claim 15, wherein the internal lumen has a shape that complements an outer shape of the first mounting post.

19. The storage system of claim 14, wherein the first mounting post is cylindrical.

20. The storage system of claim 14, further comprising the storage bin.

21. The storage system of claim 14, wherein the first mounting post is a vertical post.
